(19) United States
(12) Patent Application Publication
(10) Pub. No.: US 2006/0020274 A1
(43) Pub. Date: Jan. 26, 2006

(54) Manipulatable grasping needle

(75) Inventors: Richard C. Ewers, Fullerton, CA (US); Cang Lam, Irvine, CA (US); Vahid C. Saadat, Saratoga, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 10/898,684

(22) Filed: Jul. 23, 2004

(51) Int. Cl. A61B 17/04

(52) U.S. Cl. 606/148

(57) ABSTRACT

A manipulatable grasping needle is described herein. A piercing and grasping assembly generally comprises a needle body, which is optionally hollow, having a piercing tip and a grasping arm positioned proximally of the tip, wherein the grasping arm is adapted to project from the needle body and releasably retain a length of suture. Alternatively, opposing jaws can form a singular piercing tip when the jaws are closed. The assembly is positioned at the distal end of an elongate member which can be rigid or flexible for advancement through an endoscopic device. The elongate member can also comprise one or more articulatable sections to enable manipulation of the assembly into various shapes to facilitate suture and tissue manipulation. Moreover, either the needle body or grasping arm can define a notch for receiving suture material. A hooking member can also be provided to facilitate suture retrieval when grasping suture.

### BACKGROUND OF THE INVENTION

### FIELD OF THE INVENTION

The present invention relates to manipulatable grasping needles for suturing tissue. More particularly, the present invention relates to apparatus and methods for suturing tissue within a hollow body organ using a needle apparatus which is configured to grasp and pass sutures into or through tissue.

Various devices and methods for grasping free ends or lengths of suture and passing the suture material through tissue are known. One method generally involves attaching a suture end to a needle and passing the needle through the tissue using a needle manipulating device. Once passed through the tissue, another device is typically employed to retrieve or otherwise manipulate the needle and suture.

Other methods generally utilize sharpened needle tips which are configured to retain a suture. The needle tip is then able to penetrate the tissue and leave the suture end on the far side of the tissue where it can be grasped for further manipulation. Such grasping mechanisms generally employ various configurations such as the use of wire-like hooking elements, looped wires, etc. Some devices employ suture grasping elements, such as forceps jaws, with sharpened tips to grasp and pass the suture material.

However, many of the conventional devices have limitations in applications such as closed surgery, especially arthroscopic or laparoscopic surgery, where space and visibility constraints at the surgical site render it difficult to fully extend a suture grasping device to easily grasp a suture. Moreover, many of the devices are constrained to regions within the body accessible via straight-line access. This is typically due to the rigidity of the tool shaft upon which the graspers or needles are employed.

Furthermore, because of the typical size and rigidity of the tool, such a suture passing instrument is typically inserted within a patient as a separate tool which occupies valuable space. Additionally, other tools are typically needed to facilitate the manipulation of the suture material through the tissue.

### BRIEF SUMMARY OF THE INVENTION

Regions of tissue within a body may be secured and/or manipulated in a number of various ways. One apparatus which may be utilized particularly for piercing and/or suturing tissue while passing lengths of suture through tissue may generally comprise a needle body having a piercing tip, and a grasping arm positioned proximally of the tip wherein the grasping arm is adapted to project from the needle body and releasably retain a length of suture. Such an apparatus may be utilized either alone or it may further comprise an endoscopic device defining at least one lumen for advancing the needle body therethrough. The grasping arm may be articulated to project from the needle body to release or grasp the length of suture as desired.

The needle body and grasping arm may be positioned at the distal end of an elongate flexible member which may be tubular and may be sufficiently flexible to allow for the advancement of the needle body and elongate member through an endoscopic device. Alternatively, the elongate member may be formed as a rigid shaft for percutaneous or laparoscopic procedures. One or more sections of the elongate member may be further configured to be articulatable to conform to a desired shape. An elongate member having one or several articulatable sections may enable the assembly to be manipulated about or around tissue such that suture manipulation is facilitated.

A handle operably connected to the proximal end of the tubular member may be used to manipulate the piercing and grasping assembly into an open or closed configuration as well as articulate the assembly into a desired configuration through the use of, e.g., control wires or rods. Moreover, the elongate flexible member may be fabricated from a variety of materials, e.g., polymers, metals configured to provide flexibility, etc.

The needle body may be comprised of a needle, which may be at least partially hollow such that tissue anchor(s) may be positioned therewithin for deployment within or upon a tissue region. The needle body may have a tapered or sharpened tip for piercing into the tissue, and the needle body itself may be alternatively made of opposing tapered jaws which form a singular piercing tip when closed. As the piercing and grasping assembly is advanced into or through tissue, a length of suture may be releasably retained by the assembly between the needle body and the grasping arm, which may be positioned proximally of the tip and/or needle body or may itself form the piercing tip.

Once the piercing and grasping assembly has been desirably advanced into or through tissue, the assembly may be actuated into an open configuration where the grasping arm may project from the needle body. In the open configuration, the grasping arm may be open relative to the needle body such that the suture may be released from the piercing and grasping assembly. Alternatively, the piercing and grasping assembly may be manipulated to grasp a free length of suture. A linkage assembly, which may be actuated via a push and/or pull wire contained within the tubular member, may be used to open and close the needle body and the grasping arm. Both the needle body and grasping arm may each be actuated into an opened configuration relative to the tubular member; alternatively, the linkage assembly may be utilized to actuate a single member, i.e., needle body or grasping arm, into an opened configuration for suture manipulation or release.

Additionally, either the needle body or grasping arm, or both, may define a notch or groove to provide for clearance for suture material when retained between the needle body and grasping arm. Also, a curved or arcuate hooking member may also be provided to facilitate suture retrieval when grasping the suture.

In one illustrative use, the piercing and grasping assembly may be advanced through a tissue region or tissue fold while retaining a length of suture to be passed through. Once the assembly has been pierced through the tissue, the grasping arm and/or needle body may be articulated into an open configuration to release the suture. After the suture is freed, the assembly may be closed and withdrawn from the tissue. The assembly may then be articulated to the opposing side of the tissue and opened to receive the free suture for further manipulation. This procedure may be repeated as many times as necessary or a knot may be tied to simply secure the tissue.

To facilitate the handling of the suture, an additional needle assembly may be used in combination with one or more needle assemblies. Moreover, any variety of tools for endoluminally visualizing, grasping, plicating, manipulating, affixing, securing, etc., portions of gastric tissue may be utilized with the assembly for performing a variety of procedures.

In another example, the needle assembly and flexible elongate member may be advanced through an endoscopic device into a patient. One such procedure may entail transorally advancing the assembly through an endoscopic device into the patient's stomach to treat regions of tissue within the stomach.

### BRIEF DESCRIPTION OF THE DRAWINGS

FIG. 1 shows an example of one variation of the manipulatable grasping needle having a flexible delivery shaft.

FIG. 2A shows a detail side view of the manipulatable needle of FIG. 1 in a closed configuration for piercing tissue.

FIG. 2B shows the needle of FIG. 2A in an open configuration for releasing or grasping suture or other material.

FIG. 2C shows another variation of the manipulatable needle distal end having an articulatable portion.

FIG. 3A shows yet another variation of the manipulatable needle having a notch or groove defined in at least one of the grasping arms to accommodate a suture.

FIG. 3B shows yet another variation of the manipulatable needle having a curved or hooked portion for facilitating retrieval of a suture.

FIGS. 4A and 4B shows an example of a tissue anchor which may be positioned within the needle body and deployed from the needle body, respectively.

FIGS. 5A to 5F show an example of one use for the manipulatable needle in which the needle may be passed through a tissue fold while retaining a suture and then withdrawn from the tissue and articulated to the opposing side of tissue for additional manipulation of the suture.

FIG. 6 shows a partial cross-sectional view of another example in which an additional tool or manipulatable needle may be utilized in manipulating the suture as it is passed through the tissue fold.

FIG. 7A shows a perspective view of an example of a continuous stitch through a tissue plication which may be created utilizing a manipulatable needle.

FIG. 7B shows a perspective view of another example of how a tissue anchor may be anchored against a tissue surface to secure a tissue plication utilizing the manipulatable needle to tighten the suture.

FIG. 8 shows an illustrative view of how a manipulatable needle may be advanced through a working lumen of an endoscopic device and utilized within a hollow body organ, such as a stomach, along with an additional tool if desired.

FIGS. 9A to 9F show an example of another variation of a manipulatable needle in which a separate needle body may be deployed and/or captured via a grasper configured to allow for rotation of the needle body.

FIGS. 10A to 10D show an example of yet another variation of a manipulatable needle in which a separate needle body may be deployed and/or captured via a grasper which is configured to rotate the needle body.

FIGS. 11A to 11C show side and detail views, respectively, of yet another variation in which a suture grasper may be integrated along the body of the needle assembly.

FIG. 12A shows a side view of another variation of a manipulatable needle in which a pair of piercing jaws is positionable on a distal end of an elongate flexible member.

FIGS. 12B and 12C show side views of the needle body of FIG. 12A in which a single jaw is articulatable and in which both jaws are articulatable, respectively.

FIG. 12D shows a perspective view of the needle body of FIG. 12A.

FIG. 12E shows a side view of a variation of the needle body of FIG. 12A in which a portion of the elongate flexible member is articulatable.

FIG. 12F shows a perspective view of the needle body of FIG. 12E.

FIGS. 13A and 13B show side views of another variation of a manipulatable needle having a pivotable latch in closed and open configurations, respectively.

FIGS. 14A and 14B show side views of yet another variation of a manipulatable needle where the grasping arm may extend along a majority of the length of the needle body.

FIG. 15 shows a side view of the variation of FIGS. 14A and 14B where the elongated grasping arm may be utilized to slide over a tissue surface in retrieving a length of suture.

### DETAILED DESCRIPTION OF THE INVENTION

In creating tissue plications, a tissue plication tool having a distal tip may be advanced (transorally, transgastrically, etc.) into the stomach. The tissue may be engaged or grasped and the engaged tissue may be moved to a proximal position relative to the tip of the device, thereby providing a substantially uniform plication of predetermined size. Examples of creating and forming tissue plications may be seen in further detail in U.S. patent application Ser. No. 10/735,030 filed Dec. 12, 2003, which is incorporated herein by reference in its entirety.

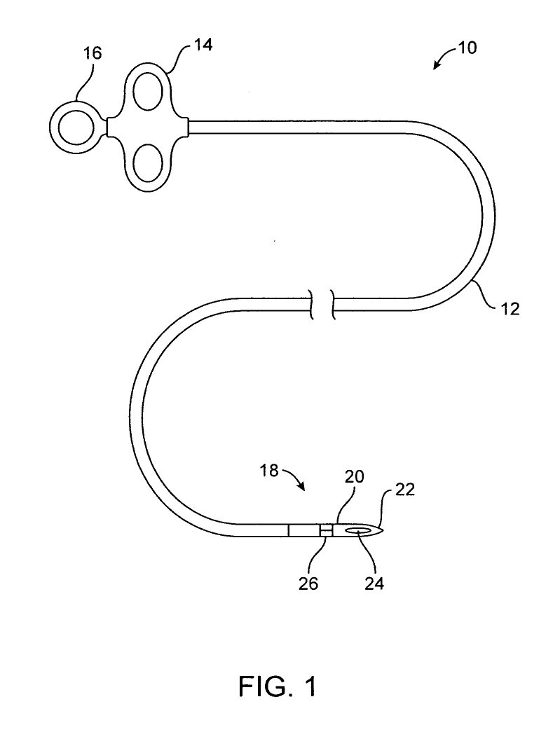

Once the tissue plication has been formed, it may be secured in a number of different ways. One apparatus which may be utilized for piercing tissue as well as passing lengths of suture through tissue may be seen in the illustrative view of FIG. 1, which shows one variation of manipulatable needle assembly 10 in a closed or piercing configuration. As shown, an elongate flexible member 12 may be tubular such that at least one lumen is defined through the length of flexible member 12. Handle 14 may be positioned at a proximal end of flexible member 12 and control handle 16 may be likewise positioned. Control handle 16 may be configured to enable the articulation of piercing and grasping assembly 18 into an open or closed configuration, as described in further detail below. Control handle 18, as well as handle 14, which is positioned at a distal end of flexible member 12, may be operably connected to piercing and grasping assembly 18, e.g., via control wires, which may run through the length of flexible member 12.

Flexible member 12 may be made from a variety of flexible materials such as polymers. If made from a polymeric material, flexible member 12 may be reinforced along its length as necessary using various methods such as interspersing metallic braids, weaves, reinforcing wires, etc., throughout the length of the flexible member 12. Alternatively, metallic materials, e.g., stainless steel, platinum, etc., and particularly superelastic metals and alloys, e.g., Nitinol, etc., may be utilized in constructing flexible member 12 provided that the material is sufficiently adapted to flex when manipulated. In the case of stainless steel or like metals, the length of flexible member 12 may be scored or perforated to allow for additional flexibility. Moreover, the diameter of flexible member 12 may be varied to suit the application in which assembly 10 may be employed. For example, if assembly 10 were advanced, e.g., through a conventional endoscope for use in a patient's stomach, flexible member may range anywhere in diameter from 2-3 mm and may have a length greater than or less than 100 cm. These dimensions are merely intended to be illustrative and are not intended to limit the size or scope of the assembly 10.

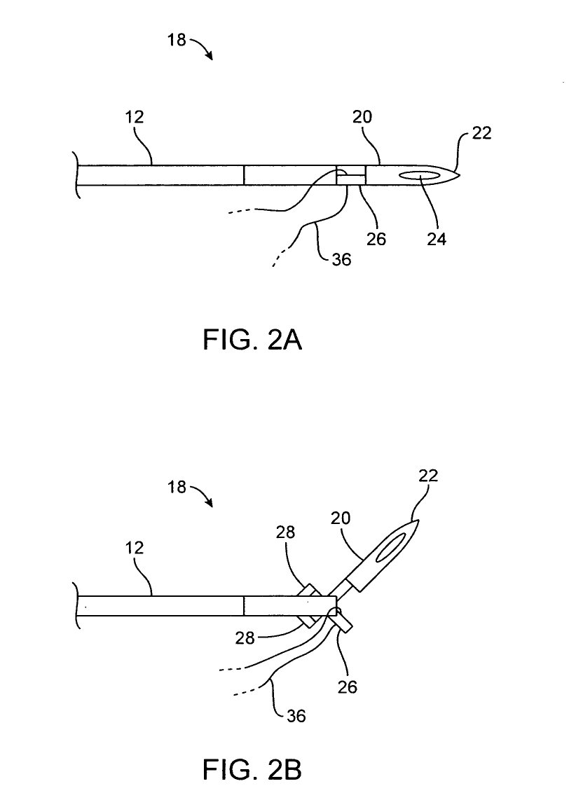

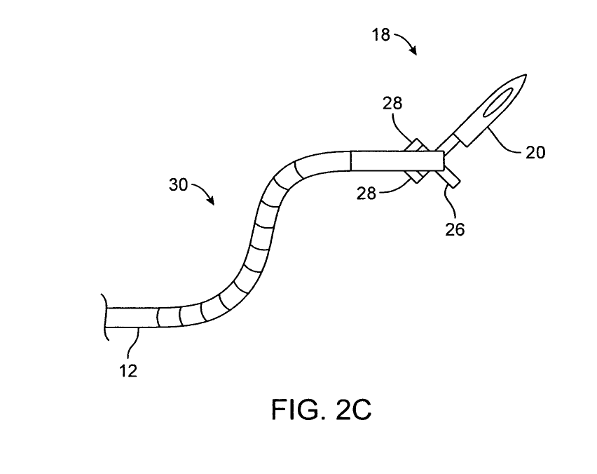

As generally shown, piercing and grasping assembly 18 may be comprised of needle body 20, which has a tapered or sharpened tip 22 for piercing into or through tissue. Needle body 20 may also define an opening or lumen 24 therethrough for retaining and passing a tissue anchor, as described further below. As seen in the detail side view of FIG. 2A, piercing and grasping assembly 18 may be configured into a low-profile closed configuration for advancement into the body and for piercing into or through tissue. As piercing and grasping assembly 18 is advanced into or through tissue, a length of suture 36 may be releasably retained by assembly 18 between needle body 20 and grasping arm 26, which may be positioned proximally of tip 22 and/or needle body 20.

Once piercing and grasping assembly 18 has been desirably advanced into or through tissue, assembly 18 may be actuated into an open configuration where grasping arm 26 may project from needle body 20, as shown in FIG. 2B. In the open configuration, grasping arm 26 may be open relative to needle body 20 such that suture 36 may be released from piercing and grasping assembly 18. Alternatively, piercing and grasping assembly 18 may be manipulated to grasp a free length of suture. Linkage assembly 28, which may be actuated via a push and/or pull wire (not shown) contained within tubular member 12, may be used to open and close needle body 20 and grasping arm 26. As shown, both needle body 20 and grasping arm 26 may each be actuated into an opened configuration relative to tubular member 12; alternatively, linkage assembly 28 may be utilized to actuate a single member, i.e., needle body 20 or grasping arm 26, into an opened configuration for suture manipulation or release.

Elongate tubular member 12 may be flexible or it may also be constructed as a rigid shaft. In either case, one or several portions of elongate member 12 may comprise an articulatable section 30 along a length of elongate member 12. A section of member 12 just proximal of piercing and grasping assembly 18 may be configured to be articulatable, as shown in FIG. 2C, such that assembly 18 may be articulated via handle 14. One or several control wires may be routed through elongate member 12 in any number of ways to enable articulatable section 30 to conform to a desired shape. An elongate member 12 having one or several articulatable sections 30 may enable assembly 18 to be manipulated about or around tissue such that suture manipulation is facilitated.

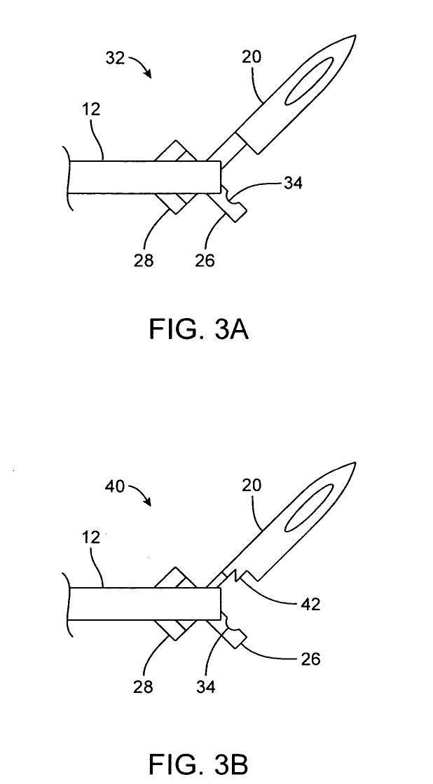

alternative needle assembly 32 is shown in the detail side view of FIG. 3A. This variation is likewise generally comprised of needle body 20 and grasping arm 26; however, a notch or groove 34 may be defined in either needle body 20, grasping arm 26, or both along the areas facing one another. Such a notch or groove 34 may be defined to provide for clearance for suture material when retained between needle body 20 and grasping arm 26.

Moreover, a suture hook or groove 42 may be further defined along either needle body 20, as shown in the alternative needle assembly 40 in FIG. 3B, along grasping arm 26, or both. Suture hook or groove 42 may simply be configured as a curved or arcuate hooking member or it may simply be configured as a protrusion. Furthermore, suture hook or groove 42 may be provided independent of or additional to notch or groove 34.

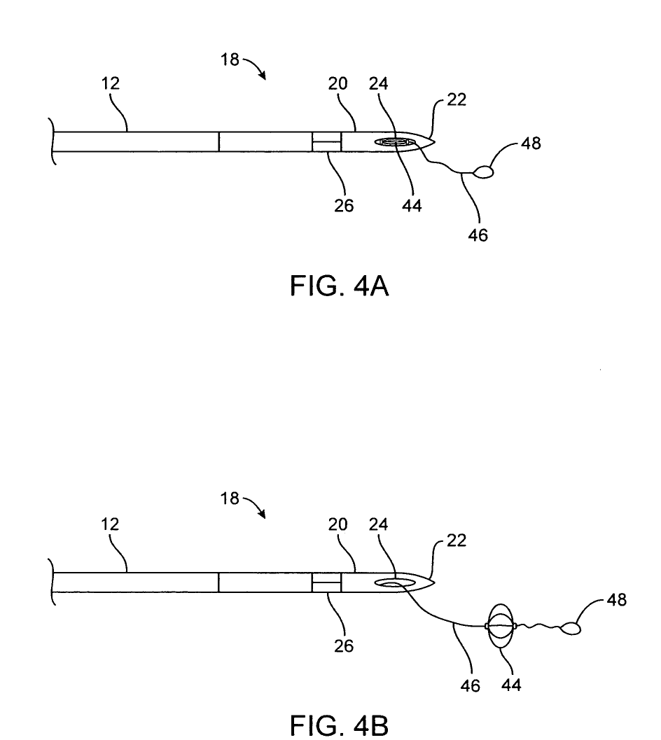

The piercing and grasping assembly 18 may be utilized in a variety of different procedures. In one instance, assembly 18 may be advanced into a hollow body organ, e.g., a stomach, and used to pierce through created tissue plications and deposit soft tissue anchors for securing the tissue plications. Examples of methods and devices for creating tissue plications may be seen in further detail in U.S. patent application Ser. No. 10/735,030 which has been incorporated by reference above. As shown in FIG. 4A, an expandable tissue anchor 44 may be seen positioned within opening 24 of needle body 20 for delivery. Suture 46 ending in terminal loop 48 may be seen passing through and from tissue anchor 44. Once assembly 18 has been desirably passed through tissue and appropriately positioned, tissue anchor 44 may be ejected from needle body 20, e.g., using a pusher mechanism. Once free from the constraints of needle body 20, tissue anchor 44 may be free to expand for anchoring against a tissue surface, as seen in FIG. 4B. Further details relating to tissue anchors and mechanisms which may be utilized for ejecting and positioning such anchors are disclosed in further detail in U.S. patent application Ser. No. 10/840,950 filed May 7, 2004, which is incorporated herein by reference in its entirety.

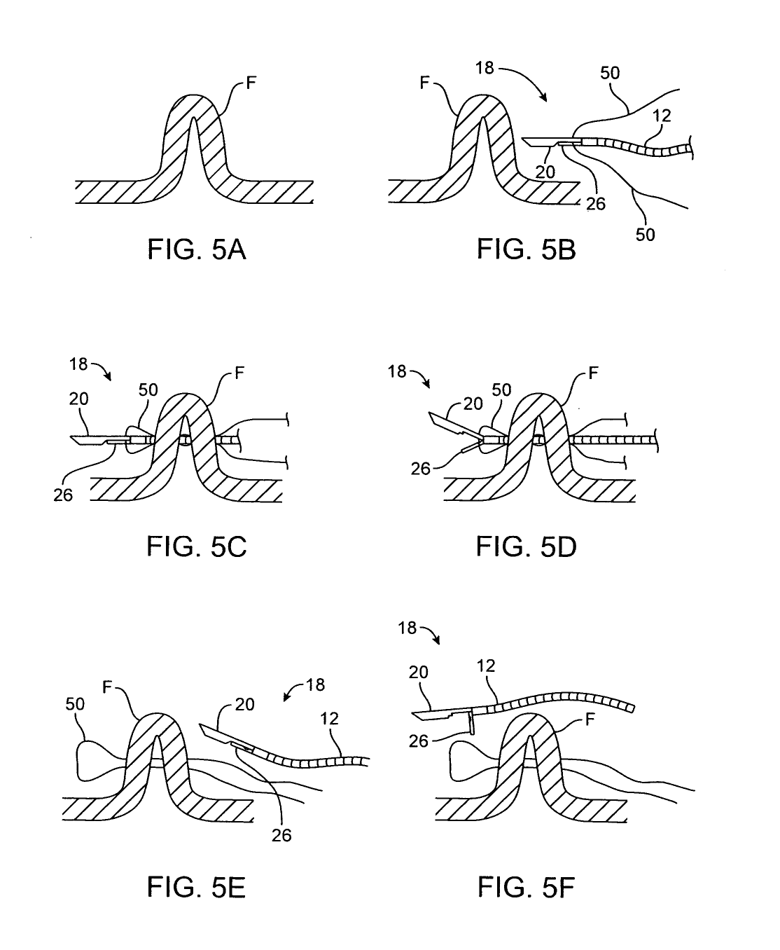

In another example of how grasping needle assembly 10 may be utilized, FIGS. 5A to 5F illustrate a partial cross-sectional view in which piercing and grasping assembly 18 may be passed through tissue fold F while retaining suture 50 and then withdrawn from the tissue fold F and articulated to the opposing side of tissue fold F for additional manipulation of suture 50. Such a procedure may be utilized to secure tissue fold F or it may be performed so that an object may be anchored within the body to the tissue fold F via anchored suture 50.

As seen in FIG. 5A, tissue fold F may be formed utilizing any number of methods described above or otherwise conventionally known. Piercing and grasping assembly 18 may be advanced towards tissue fold F while releasably retaining a length of suture 50 to be deposited through tissue fold F. Elongate tubular member 12 may comprise a flexible member and/or it may also comprise a rigid shaft, depending upon the desired procedure to be performed. Moreover, tubular member 12 may further comprise one or more articulatable sections to facilitate manipulation of piercing and grasping assembly 18 about or around tissue fold F.

Once assembly 18 has been pierced through tissue fold F, as shown in FIG. 5C, grasping arm 26 and/or needle body 20 may be articulated into an open configuration to release suture 50 from assembly 18 on the distal side of tissue fold F, as shown in FIG. 5D. After suture 50 has been freed from assembly 18, grasping arm 26 and/or needle body 20 may be configured into its closed configuration and withdrawn from tissue fold F, as shown in FIG. 5E. Assembly 18 may then be articulated to the opposing distal side of tissue fold F and opened to receive the free suture 50 for further manipulation, as shown in FIG. 5F. This procedure may be repeated or a knot may be tied to simply secure the tissue fold F.

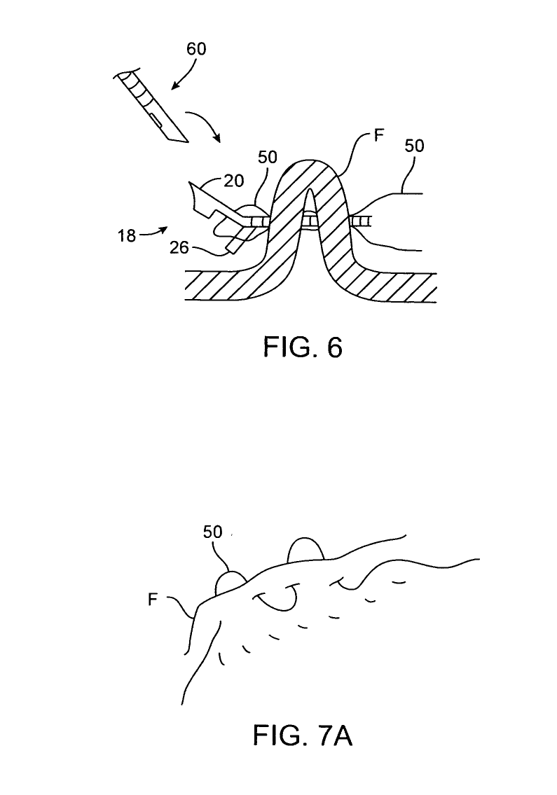

To facilitate the handling of suture 50, an additional needle assembly 60 may be used in combination with one or more needle assemblies 18, as shown in the partial cross-sectional view of FIG. 6. Moreover, any variety of tools for endoluminally visualizing, grasping, plicating, manipulating, affixing, securing, etc., portions of gastric tissue may be utilized with the assembly 18 for performing a variety of procedures. Other examples of applicable tools may be seen in U.S. patent application Ser. Nos. 10/734,547 and 10/734,562, both filed Dec. 12, 2003 and both incorporated herein by reference in their entirety. Other examples of various tools which may be utilized are also further described in U.S. patent application Ser. No. 10/639,162 filed Aug. 11, 2003 and 10/672,375 filed Sep. 26, 2003, each of which is also incorporated herein by reference in its entirety.

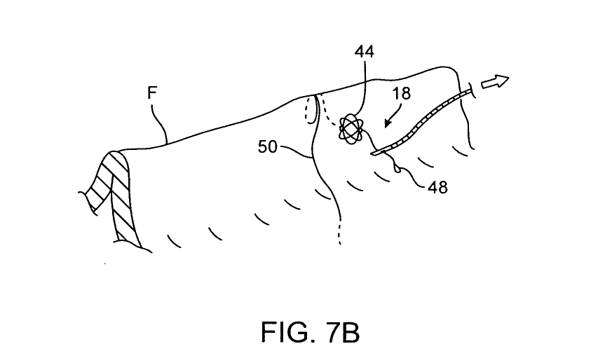

FIG. 7A shows a perspective view of tissue fold F with suture 50 in a running stitch to secure the plication as one example of a potential procedure which may be accomplished utilizing assembly 18 either alone or in combination with any one of the above-mentioned tools. FIG. 7B shows a perspective view of tissue fold F with suture 50 utilized with tissue anchor 44 in securing tissue fold F as yet another example. As mentioned above for FIGS. 4A and 4B, assembly 18 may be passed through tissue fold F and then utilized to deploy tissue anchor 44 from the needle body. Once tissue anchor 44 has been deployed, assembly 18 may be manipulated to knot suture 50 about tissue fold F. Assembly 18 may then be further manipulated to grasp a free end of suture 50, e.g., at suture terminal end 48, and suture 50 may be pulled or tensioned via assembly 18 in the direction of the arrow shown such that tissue anchor 44 becomes drawn securely against the tissue surface of fold F. Other variations or modifications for knotting or securing suture 50 and/or tissue anchor (or tissue anchors) 44 against tissue fold F are intended to be included in the scope of this disclosure and the claims below.

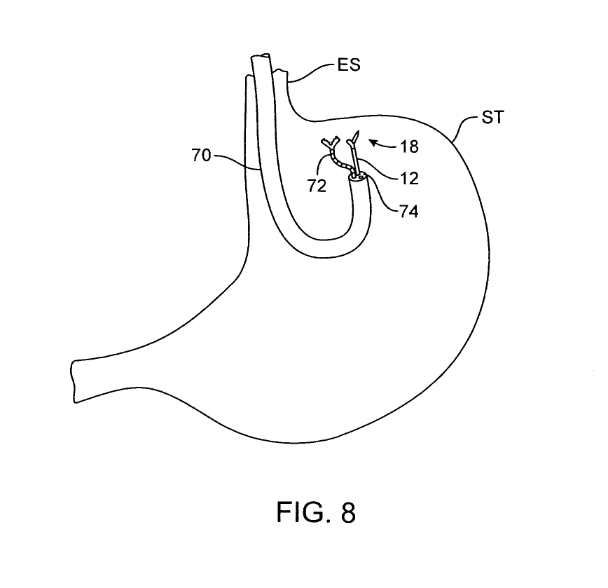

In another example for utilizing grasping needle assembly 10, FIG. 8 shows an illustrative view of how a flexible tubular member 12 (optionally with or without an articulatable section) and assembly 18 may be advanced through a working lumen 74 of an endoscopic device 70. As illustrated, endoscopic device 70, which may comprise a conventional endoscope or a rigidizable endoscope, may be advanced transorally through a patient's esophagus ES and into a stomach ST. Once the distal portion of endoscopic device 70 has been desirably positioned, assembly 18 may be advanced through a working lumen 74 optionally with one or more additional tools 72 having end effectors in adjacent lumens for performing any number of procedures upon the tissue.

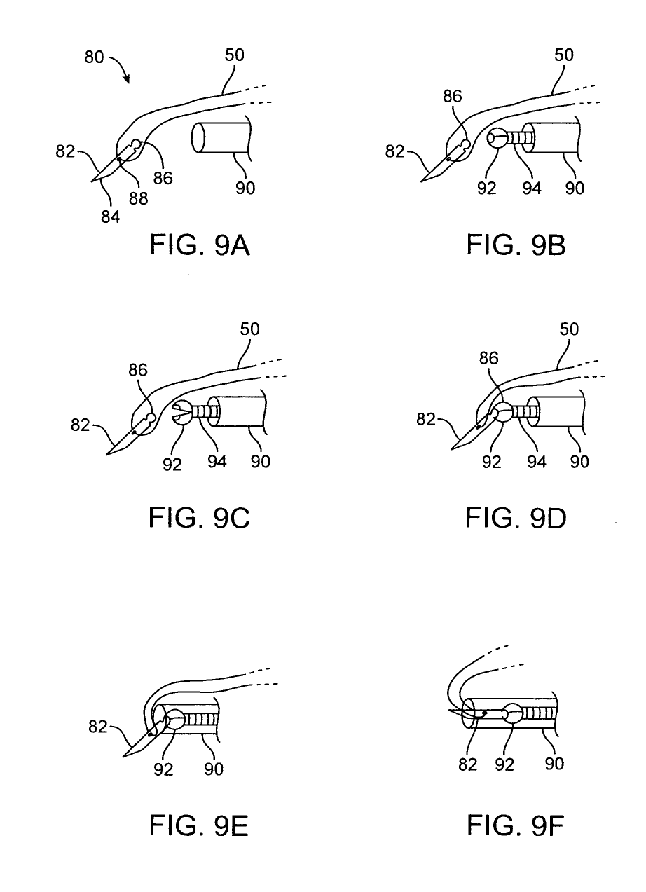

FIGS. 9A to 9F show an alternative needle assembly 80 which may be utilized in a manner similar to that described above. Generally, assembly 80 may comprise a separate needle body 82 having a tapered or sharpened tip 84 for piercing into and/or through tissue. An opening 88 may be defined through needle body 82 for passage of suture 50 therethrough. A proximal end of needle body 82 may be configured into a rounded sphere-like member 86 to facilitate the grasping and retrieval of needle body 82.

In use, once needle body 82 has been passed through tissue via delivery tube or catheter 90, needle body 82 may be released to enable delivery tube 90 to be articulated about the tissue. To recapture needle body 82, delivery tube 90 may be positioned adjacent to proximal end 86 of needle body 82, as shown in FIG. 9A. Once delivery tube 90 has been desirably positioned, grasper 92 may be advanced out of delivery tube 90 via elongate member 94, as shown in FIG. 9B. Grasper 92 may be configured to form spherically-shaped grasping arms which may be clamped over proximal end 86 in a complementary pivoting relationship. Proximal end 86 may then be securely grasped via grasper 92, as shown in FIGS. 9C and 9D. As grasper 92 is withdrawn within delivery tube 90, as shown in FIGS. 9E and 9F, needle body 82 may be forced to automatically rotate about proximal end 86 into a straightened configuration relative to delivery tube 90.

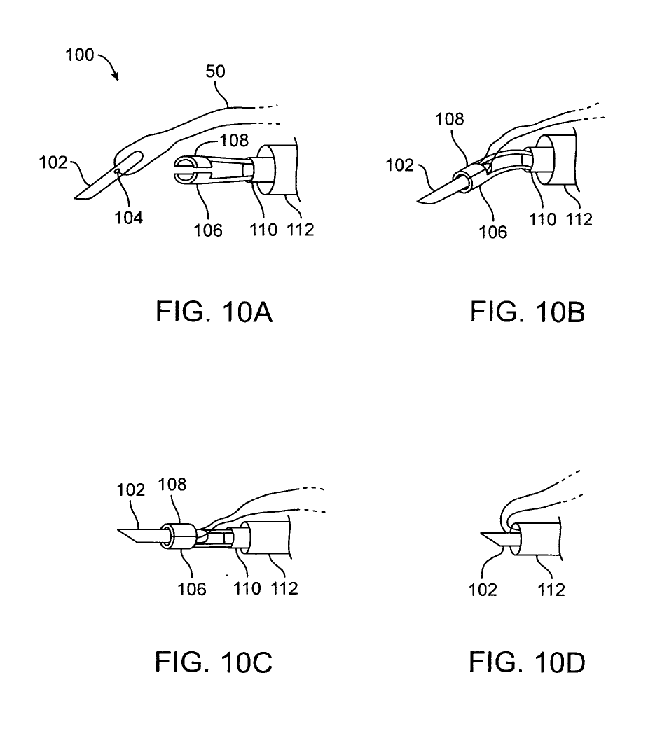

Another variation is shown in needle assembly 100, generally comprising needle body 102 which defines opening 104 for passage of suture 50 therethrough. Two or more articulatable grasping arms 106, 108 may be positioned to extend from an elongate member 110. In operation, assembly 100 may be utilized in the same or similar manner as assembly 80 above. Once needle body 102 is to be grasped, delivery tube or catheter 112 may be advanced adjacent to needle body 102. In this variation, grasping arms 106, 108 may be articulated to become angled relative to elongate member 110 to facilitate the grasping of needle body 102, as shown in FIGS. 10A and 10B. Once needle body 102 has been securely grasped, grasping arms 106, 108 may be rotated or straightened such that needle body 102 is aligned in parallel with delivery tube or catheter 112, as shown in FIG. 10C. Grasping arms 106, 108 may be articulated, e.g., via control wires routed through elongate member 110. Once aligned, needle body 102 may then be withdrawn proximally into delivery tube or catheter 112 to retrieve not only the needle but also suture 50. Needle body 102 may be further articulated and passed through another area of tissue or another procedure, as desired, may be performed.

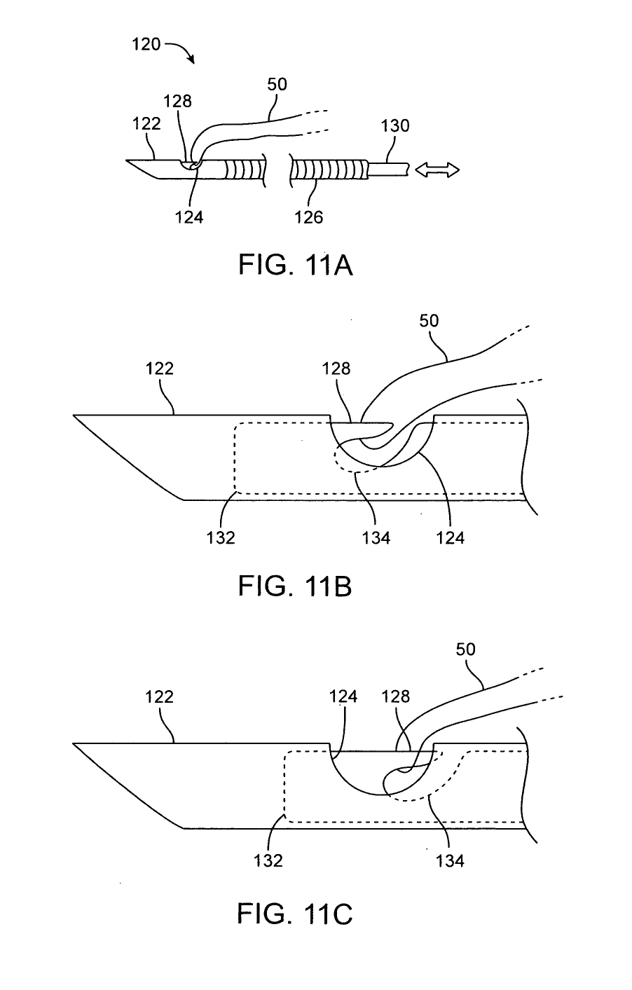

Yet another variation for a grasping needle assembly is shown in the illustrative side view of needle assembly 120 in FIG. 11A. This variation may generally comprise needle body 122 which defines a lumen at least partially therethrough. An opening or port 124 may be defined along a surface of needle body 122 within which suture 50 may be releasably retained. Needle body 122 may be positioned at a distal end of tubular member 126, which may be flexible along its length and/or comprise one or more articulatable sections.

An inner member or block 132 may be slidably positioned within needle body 122 such that a portion of block 132 which defines a suture-receiving notch or groove 134 passes adjacent to opening or port 124, as shown in the detail view in FIG. 11B. Notch or groove 134 may be defined along block 132 such that a hooked member 128 is formed distally of notch 134. To actuate block 132 to slide longitudinally, an inner tubular member or control rod 130 may be routed through tubular member 126 and control rod 130 may be manipulated via its proximal end to slide block 132 back and forth.

In use, notch or groove 134 may be aligned with opening 124, as shown in FIG. 11B, such that a length of suture 50 may be positioned within notch or groove 134. To secure suture 50 to needle body 122, control rod 130 may be actuated proximally such that block 132 is pulled proximally and suture 50 is held between hooked member 128 and an edge of opening 124, as shown in FIG. 11C. Needle body 122, along with suture 50, may thus be passed through tissue. To release suture 50 from needle body 122, block 132 may be translated distally to thus release suture 50 from between hooked member 128 and opening 124.

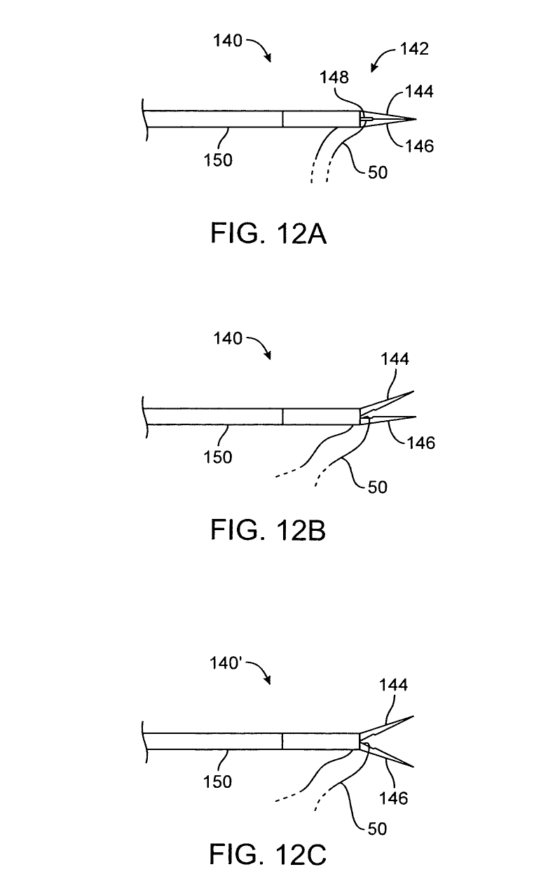

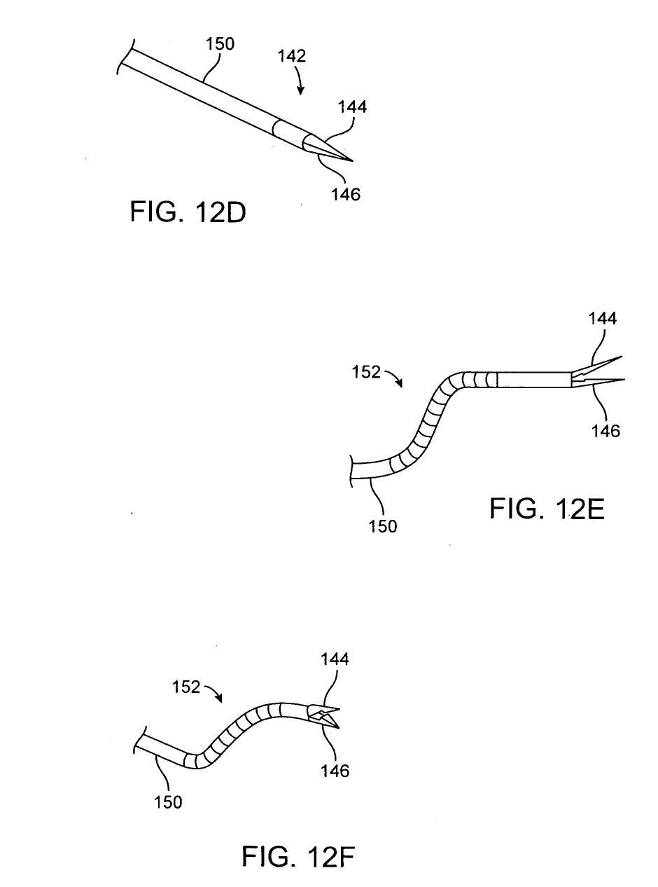

Yet another needle assembly variation is shown in the side view of FIG. 12A in needle assembly 140. Piercing and grasping assembly 142 may be positioned at a distal end of elongate flexible member 150 and may generally comprise opposing jaws, e.g., upper and lower grasping arms 144, 146, respectively, which are articulatable between an open and closed configuration. Each of the grasping arms 144, 146 may define a tapered or sharpened tip such that when grasping arms 144, 146 are in a closed configuration, a singular piercing tip is formed to facilitate entry into or through tissue. A notch or groove 148 may be formed in one or both grasping arms 144, 146 to accommodate a suture 50 when releasably held therebetween.

In articulating grasping arms 144, 146 into its open configuration when grasping or releasing suture 50, one or both grasping arms 144, 146 may be articulated to open relative to one another. For instance, FIG. 12B shows a variation 140 where a single grasping arm 144 may be articulated to open. FIG. 12C shows another variation 140′ in which both grasping arms 144, 146 may be articulated to open. FIG. 12D shows a perspective view of grasping assembly 142 and the piercing tip formed by grasping arms 144, 146 positioned upon the distal end of flexible member 150.

FIG. 12E shows another variation in which flexible tubular member 150 may comprise one or more articulatable sections 152. The operation of articulatable section 152 is similar to that described above. FIG. 12F shows a perspective view of grasping arms 144, 146 along with articulatable section 152.

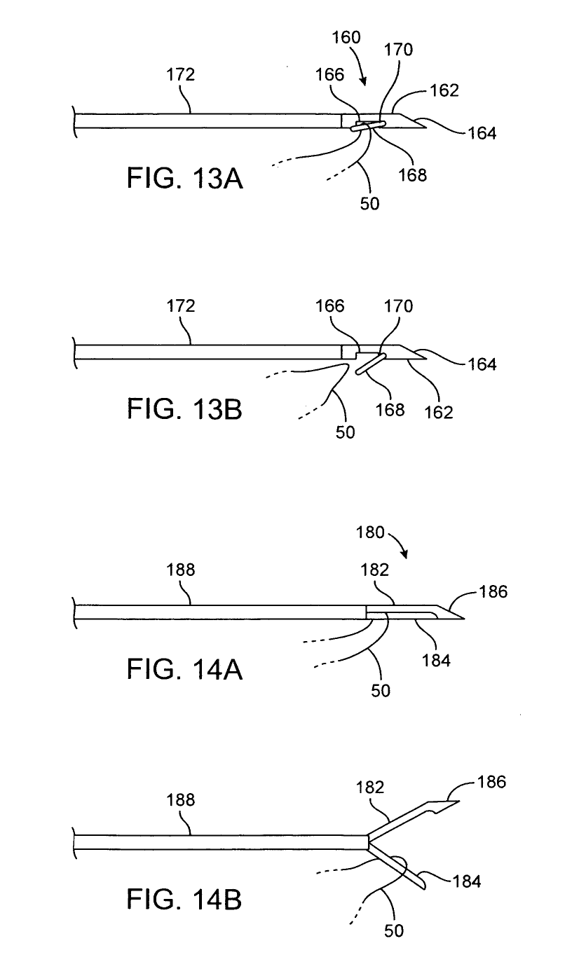

FIGS. 13A and 13B show side views of yet another variation of the piercing and grasping assembly 160 in closed and open configurations. In this variation, assembly 160 may comprise needle body 162 having a tapered piercing tip 164. Suture retaining notch or groove 166 may be defined along the length of needle body 162, preferably on a side of needle body 162 opposite to the tapered piercing tip 164. As shown in FIG. 13A, grasping or retaining arm 168 is shown in a closed configuration while retaining suture 50. To release or to grasp suture 50, as shown in FIG. 13B, arm 168 may be opened, e.g., via pivot 170, located on a distal end of arm 168 such that arm 168 opens proximally of needle body 162. To grasp suture 50, needle body 162 may be pulled proximally via elongate tubular member 172 to scoop up suture 50.

FIGS. 14A and 14B show yet another variation in piercing and grasping assembly 180 in which a length of elongated grasping arm 184 may be such that it resides along a majority of a length of needle body 182. As seen in the closed configuration in FIG. 14A, grasping arm 184 may have a length which resides along needle body 182 preferably opposite to tapered piercing end 186. Assembly 180 may be manipulated via elongate tubular member 188. When grasping arm 184 is opened, as shown in FIG. 14B, the lengthened arm 184 may facilitate the grasping of suture 50.

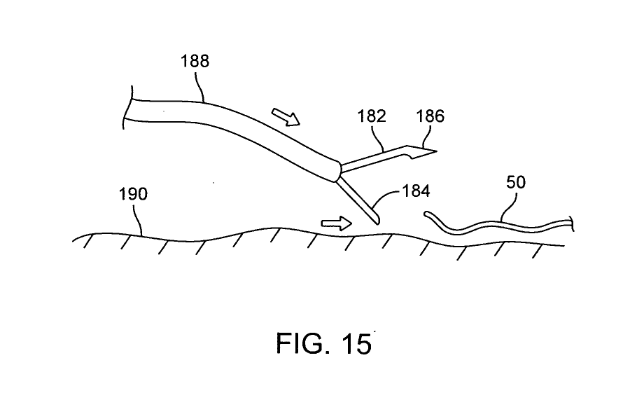

Moreover, retrieval of suture 50 may be facilitated by having grasping arm 184 define an atraumatic distal tip. When retrieving suture 50 from a surface of tissue 190, elongate tubular member 188 may be advanced distally while allowing grasping arm 184 to slide over tissue surface 190, as shown by the arrows in FIG. 15, to facilitate grasping suture 50 from the surface.

Although a number of illustrative variations are described above, it will be apparent to those skilled in the art that various changes and modifications may be made thereto without departing from the scope of the invention. Moreover, although specific configurations and applications may be shown, it is intended that the manipulatable needles, endoscopic devices, etc., may be utilized in various types of procedures in various combinations as practicable. It is intended in the appended claims to cover all such changes and modifications that fall within the true spirit and scope of the invention.

What is claimed is:

1. An apparatus for suturing tissue comprising:
a needle body having a piercing tip; and
a grasping arm positioned proximally of the tip,
the grasping arm being adapted to project from the needle body and releasably retain a length of suture.

2. The apparatus of claim 1 further comprising an elongate flexible member connected to the needle body.

3. The apparatus of claim 2 wherein the elongate flexible member comprises a hollow tubular member.

4. The apparatus of claim 2 wherein the elongate flexible member comprises an articulatable section proximally of the needle body.

5. The apparatus of claim 1 further comprising a control handle operably connected to the grasping arm and adapted to articulate the grasping arm between an open and closed configuration.

6. The apparatus of claim 1 wherein the needle body defines a lumen therethrough.

7. The apparatus of claim 6 wherein the needle body is adapted to retain a tissue anchor therein for delivery into or through tissue.

8. The apparatus of claim 1 wherein the grasping arm is articulatable via a linkage between an open and closed configuration.

9. The apparatus of claim 1 wherein the grasping arm or needle body defines a notch for receiving the suture therein.

10. The apparatus of claim 1 wherein the grasping arm or needle body comprises a hook for retaining the suture.

11. The apparatus of claim 1 wherein the grasping arm is adapted to project from the needle body via a pivot positioned proximally of the grasping arm.

12. The apparatus of claim 1 wherein the grasping arm is adapted to project from the needle body via a pivot positioned distally of the grasping arm.

13. The apparatus of claim 1 wherein the grasping arm has a length along a majority of a length of the needle body.

14. The apparatus of claim 13 wherein the grasping arm comprises an atraumatic distal end.

15. A system for suturing tissue comprising:
a needle body having a piercing tip and a grasping arm positioned proximally of the tip, the grasping arm being adapted to project from the needle body and releasably retain a length of suture; and
an endoscopic device defining at least one lumen for advancing the needle body therethrough.

16. The system of claim 15 further comprising a tool having an end effector for advancing through the endoscopic device adjacent the needle body.

17. The system of claim 15 further comprising an elongate flexible member connected to the needle body.

18. The system of claim 17 wherein the elongate flexible member comprises a hollow tubular member.

19. The system of claim 17 wherein the elongate flexible member comprises a hollow tubular member.

20. The system of claim 17 wherein the elongate flexible member comprises an articulatable section proximally of the needle body.

21. The system of claim 15 further comprising a control handle operably connected to the grasping arm and adapted to articulate the grasping arm between an open and closed configuration.

22. The system of claim 15 wherein the needle body defines a lumen therethrough.

23. The system of claim 15 wherein the needle body is adapted to retain a tissue anchor therein for delivery into or through tissue.

24. The system of claim 15 wherein the grasping arm is articulatable via a linkage between an open and closed configuration.

25. The system of claim 15 wherein the grasping arm or needle body defines a notch for receiving the suture therein.

26. The system of claim 15 wherein the grasping arm or needle body comprises a hook for retaining the suture.

27. The system of claim 15 wherein the endoscopic device comprises an articulatable distal portion.

28. The system of claim 15 wherein the endoscopic device comprises a flexible length.

29. The system of claim 15 wherein the endoscopic device comprises a rigidizable length.

30. An apparatus for suturing tissue comprising:
an elongate flexible member having a proximal end, a distal end, and a length therebetween;
a grasper having opposing jaws positioned on the elongate member distal end;
wherein the opposing jaws are articulatable between open and closed configurations for releasably retaining a length of suture therebetween, and
wherein the opposing jaws define a piercing tip in the closed configuration for passage into or through tissue.

31. The apparatus of claim 30 wherein the elongate flexible member comprises a hollow tubular member.

32. The apparatus of claim 30 wherein the elongate flexible member comprises an articulatable section proximally of the grasper.

33. The apparatus of claim 30 further comprising a control handle operably connected to the grasper and adapted to articulate the grasper between the open and closed configurations.

34. The apparatus of claim 30 further comprising an endoscopic device defining at least one lumen for advancing the grasper therethrough.

35. The apparatus of claim 34 wherein the endoscopic device comprises a flexible length.

36. The apparatus of claim 34 wherein the endoscopic device comprises a rigidizable length.

37. The apparatus of claim 30 wherein the grasper defines a notch or groove in one or both opposing jaws for receiving the length of suture therein when the opposing jaws are in the closed configuration.

38. A method for passing a length of suture into or through tissue, comprising:
advancing a needle body having a piercing tip and a grasping arm positioned proximally of the tip through a first region of tissue; and
articulating the grasping arm to project from the needle body and release the length of suture on a second region of tissue distal to the first region.

39. The method of claim 38 wherein advancing a needle body having a piercing tip comprises advancing the needle body within a hollow body organ.

40. The method of claim 38 wherein advancing a needle body having a piercing tip comprises piercing the first region of tissue via the tip.

41. The method of claim 38 further comprising retracting the grasping arm and withdrawing the needle body proximally through the tissue.

42. The method of claim 41 further comprising advancing the needle body to the second region of tissue and grasping the length of suture with the grasping arm.

43. The method of claim 42 further comprising advancing the needle body while retaining the length of suture through a third region of tissue.

44. The method of claim 43 wherein the third region of tissue is adjacent to the first region of tissue.

45. The method of claim 43 wherein the third region of tissue is distal to the first region of tissue.

46. The method of claim 38 further comprising grasping the length of suture via a second needle body having a piercing tip and a grasping arm.

47. The method of claim 38 further comprising manipulating the tissue into a plication prior to advancing a needle body.

48. The method of claim 38 wherein advancing a needle body comprises advancing the needle body via an elongate flexible member.

49. The method of claim 48 further comprising articulating the needle body relative to the elongate flexible member.

50. The method of claim 38 wherein advancing a needle body comprises advancing the needle body through an endoscopic device.

51. The method of claim 50 further comprising rigidizing the endoscopic device.

52. A method for suturing tissue comprising:
advancing a needle body having a piercing tip and a grasping arm positioned proximally of the tip through a first region of tissue;
articulating the grasping arm to project from the needle body and release a length of suture on a second region of tissue distal to the first region;
retracting the grasping arm and withdrawing the needle body proximally through the tissue;
advancing the needle body to the second region of tissue;
grasping the length of suture with the grasping arm; and
advancing the needle body while retaining the length of suture through a third region of tissue.
